(19) United States
(12) Patent Application Publication
(10) Pub. No.: US 2019/0167415 A1
(43) Pub. Date: Jun. 6, 2019

(54) SURGICAL TOOL AND METHOD

(71) Applicants: Theodore Todd Richmond, Doylestown, PA (US); Albert Jun, Towson, MD (US); Jan McGlumphy, Bel Air, MD (US)

(72) Inventors: Theodore Todd Richmond, Doylestown, PA (US); Albert Jun, Towson, MD (US); Jan McGlumphy, Bel Air, MD (US)

(21) Appl. No.: 16/210,683

(22) Filed: Dec. 5, 2018

Related U.S. Application Data
(60) Provisional application No. 62/594,848, filed on Dec. 5, 2017.

(51) Int. Cl. A61F 2/14; A61F 9/007; A61B 17/29

(52) U.S. Cl. CPC A61F 2/148; A61F 2/142; A61B 2017/00969; A61B 17/2909; A61F 9/007

(57) ABSTRACT

A surgical tool assembly is disclosed. The surgical tool assembly includes a forceps instrument including a proximal handle portion with an actuator, and a distal forceps portion including jaws. The actuator selectively moves the jaws. The surgical tool assembly includes an infusion assembly including a tube configured to be connected to a fluid source at a primary tube end and connected to a fitting at a paracentesis tube end. The fitting defines a passage configured to receive the distal forceps portion of the forceps instrument. An infusion sleeve extends away from the fitting. The infusion sleeve is arranged concentric around an outer surface of the distal forceps portion to define a fluid pathway. The jaws extend beyond a terminal end of the infusion sleeve such that the jaws are arranged outside of the infusion sleeve.

### INCORPORATION BY REFERENCE

The following document is incorporated by reference as if fully set forth herein: U.S. Provisional Application 62/594,848, filed Dec. 5, 2017.

### FIELD OF INVENTION

The present invention generally relates to a medical device, and more particularly relates to a surgical tool.

### BACKGROUND

Ophthalmic surgery requires precise tools and reliable methods to minimize trauma to the anterior chamber of the patient's eye. One type of ophthalmic surgery is Descemet membrane endothelial keratoplasty (DMEK) surgery. DMEK is a cornea transplant procedure that involves selective removal of the patient's Descemet membrane and endothelium, without any stroma, followed by the transplant of donor corneal endothelium and Descemet membrane donor tissue. DMEK is considered a partial thickness corneal transplant surgery which primarily replaces the innermost portion of the cornea rather than the full thickness of the cornea. DMEK surgery has the advantage of providing more rapid visual recovery, lower donor tissue rejection rates, and better refractive outcomes than procedures using thicker corneal donor tissue containing the stroma and other corneal layers. The donor tissue for DMEK surgery is considerably thinner in comparison to tissues used in other corneal transplant procedures, measuring approximately 10-15 microns thick. Because the donor tissue is thin and has no stroma attached, it is susceptible to being flimsy and to scrolling upon itself, which creates a challenge when attempting to prepare, handle, transfer, and properly place and position the donor tissue in the anterior chamber of the patient's eye.

Known methods for performing this surgery include injecting a donor tissue into the patient's eye through a single port. As shown in FIG. 1, one type of surgery according to the prior art involves a relatively larger gauge tool 1 that injects a donor tissue into the eye 2 via a single port 3. This type of surgery requires a relatively larger incision, typically 3.2-4.0 mm in length, to accommodate injecting the donor tissue. This larger incision may require the application of sutures in order to be closed. Other known methods and tools for DMEK surgery do not provide a reliable configuration for orienting and unscrolling a donor tissue once implanted in a patient's eye. Known methods for promoting unscrolling and orientation of the donor tissue, such as repeated tapping of the patient's eye, twisting the insertion devices, etc., are time consuming, less reliable, and may lead to damage of the donor tissue.

Accordingly, there is a need for a reliable surgical tool and method that is less invasive, more efficient for unscrolling the donor tissue, and reduces the chance of causing trauma to the patient's eye and the donor tissue during the procedure.

### SUMMARY

Briefly stated, a surgical tool assembly and a surgical method are disclosed that reliably, quickly, and correctly orients, unscrolls, and accurately places a donor tissue implanted into the anterior chamber of a patient's eye.

The surgical tool assembly includes a forceps instrument including a proximal handle portion with an actuator, and a distal forceps portion including jaws. The actuator selectively moves the jaws between an open position and a closed or gripped position. The surgical tool assembly includes an infusion assembly including a tube configured to be connected to a fluid source at a primary (first) tube end and connected to a fitting at a paracentesis (second) tube end. The fitting defines a passage configured to receive the distal forceps portion of the forceps instrument. An infusion sleeve extends away from the fitting. The infusion sleeve is arranged concentric around an outer surface of the distal forceps portion to define a fluid pathway. The jaws extend beyond a terminal end of the infusion sleeve such that the jaws are arranged outside of the infusion sleeve.

A method of performing corneal surgery is also disclosed. The method includes providing a surgical tool assembly comprising a forceps instrument including a proximal handle portion with an actuator, and a distal forceps portion including jaws. The actuator moves the jaws between an open position and a closed or gripped position. The surgical tool assembly includes an infusion assembly including a tube configured to be connected to a fluid source at a primary tube end and connected to a fitting at a paracentesis tube end. An infusion sleeve extends away from the fitting, and the fitting defines a passage configured to receive the distal forceps portion of the forceps instrument. The infusion sleeve is arranged concentric around an outer surface of the distal forceps portion to define a fluid pathway. The jaws extend beyond a terminal end of the infusion sleeve such that the jaws are arranged outside of the infusion sleeve. A donor tissue preparation tool (a holder) is configured to support a donor tissue. The donor tissue will be positioned in a scrolled orientation (a scroll) on the donor preparation tool with the endothelial surface facing inward. The method includes cutting two incisions on the anterior chamber of the patient's eye. The method includes placing the donor tissue on the donor tissue preparation tool and positioning the donor tissue preparation tool adjacent to the primary incision (a 2.0-2.5 mm incision). The method includes inserting the distal forceps portion of the forceps instrument through the paracentesis incision (a 1.0-1.5 mm incision), through the anterior chamber of the patient's eye and out of the primary incision. The method includes securing the donor tissue from the donor tissue preparation tool via the jaws and withdrawing the distal forceps portion back through the primary incision and into the anterior chamber of the patient's eye at the implant site. Finally, the method includes providing pressurized fluid to the fluid pathway of the infusion sleeve while gripping the donor tissue to orient, unscroll, and accurately place the donor tissue at the implant site. While holding the donor tissue in position, a small amount of fluid is aspirated to shallow, i.e. to decrease the volume of, the anterior chamber of the patient's eye to prevent the donor tissue from scrolling.

Additional features of the surgical tool assembly and the method are described in more detail below.

### BRIEF DESCRIPTION OF THE DRAWINGS

The following drawings are illustrative of preferred embodiments of the present invention, and are not intended to limit the invention as encompassed by the claims forming part of the application, wherein like items are identified by the same reference designations:

FIG. 1 is an illustration of a surgical tool and method according to the prior art.

FIG. 2A is a perspective view of a surgical tool assembly according to one embodiment in an assembled state.

FIG. 2B is a magnified view of the surgical tool assembly of FIG. 2A.

FIG. 3 is a magnified view of the surgical tool assembly in a disassembled state.

FIG. 4 is a perspective view of a forceps instrument and an infusion assembly of the surgical tool assembly.

FIG. 5 is a magnified view of the forceps instrument and the infusion assembly of the surgical tool assembly.

FIGS. 6A-6F illustrate steps for performing a surgical procedure according to one embodiment.

FIG. 7A illustrates an alternative arrangement of the surgical tool assembly.

FIG. 7B is a magnified view of the infusion assembly of FIG. 7A.

FIG. 7C is a magnified view of the forceps instrument of FIG. 7A.

FIG. 7D is an alternative view of the surgical tool assembly of FIG. 7A.

FIG. 7E is a magnified view of an interface of the forceps instrument and infusion assembly of FIG. 7A.

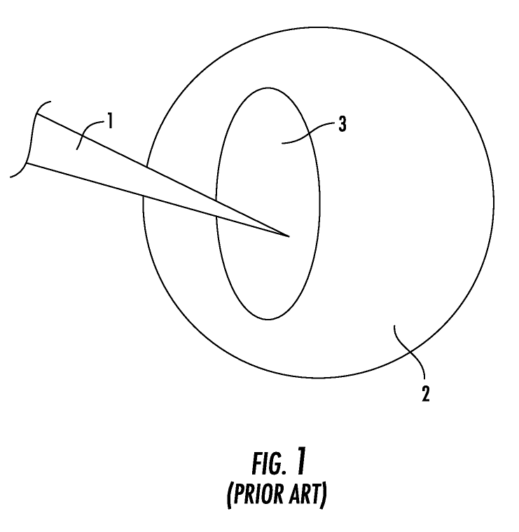

### DETAILED DESCRIPTION OF THE PREFERRED EMBODIMENTS

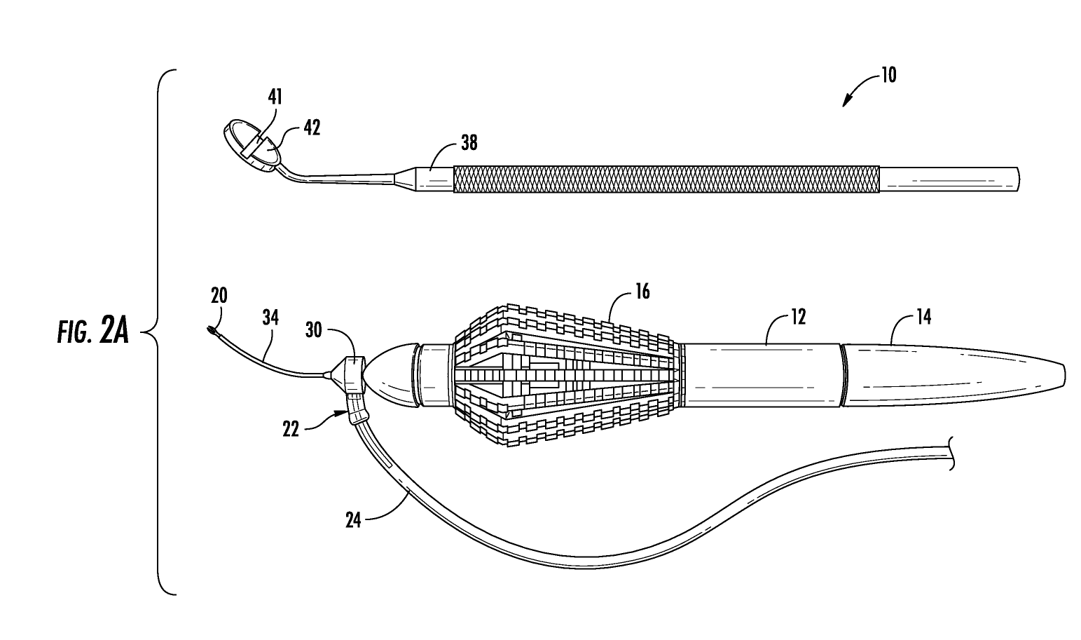

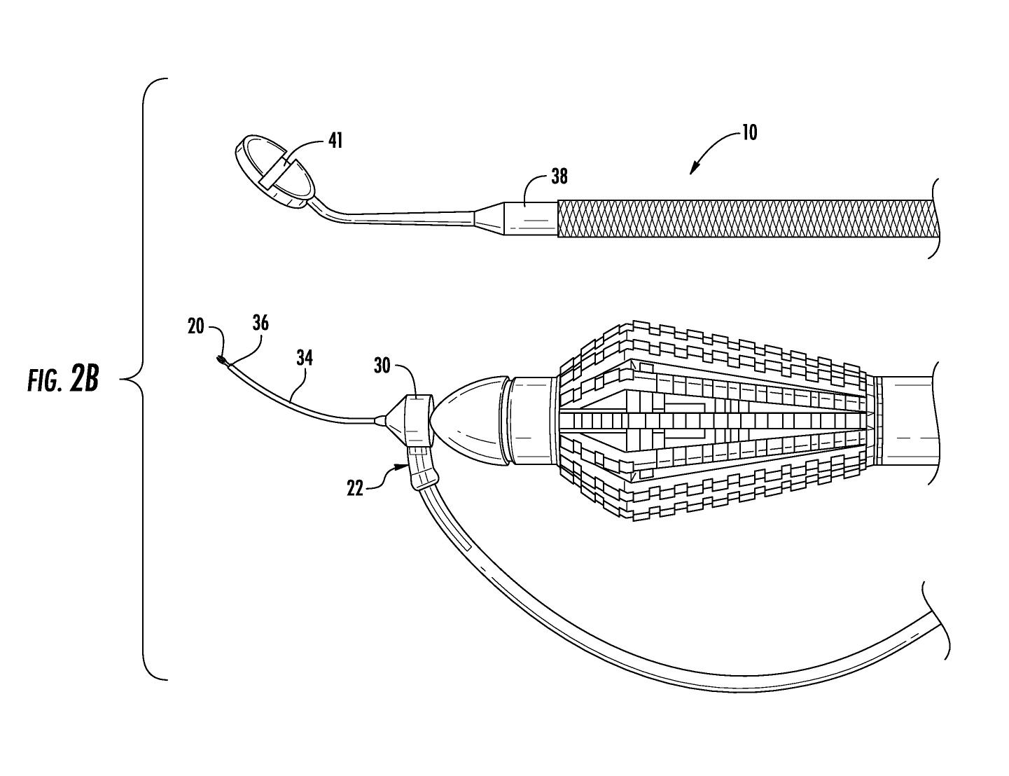

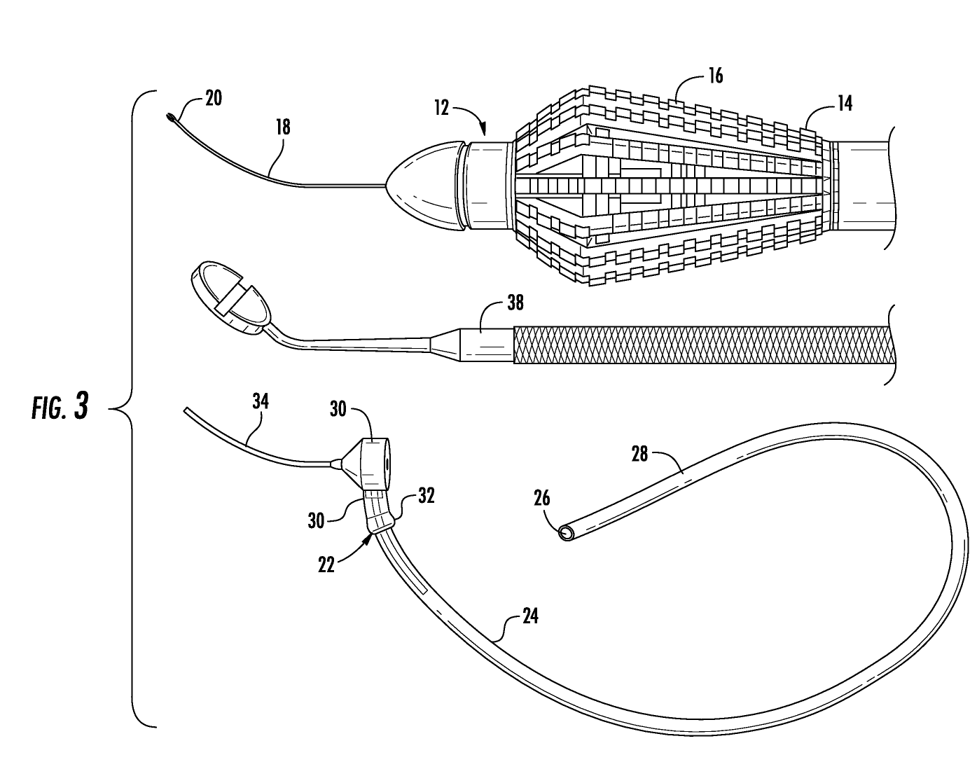

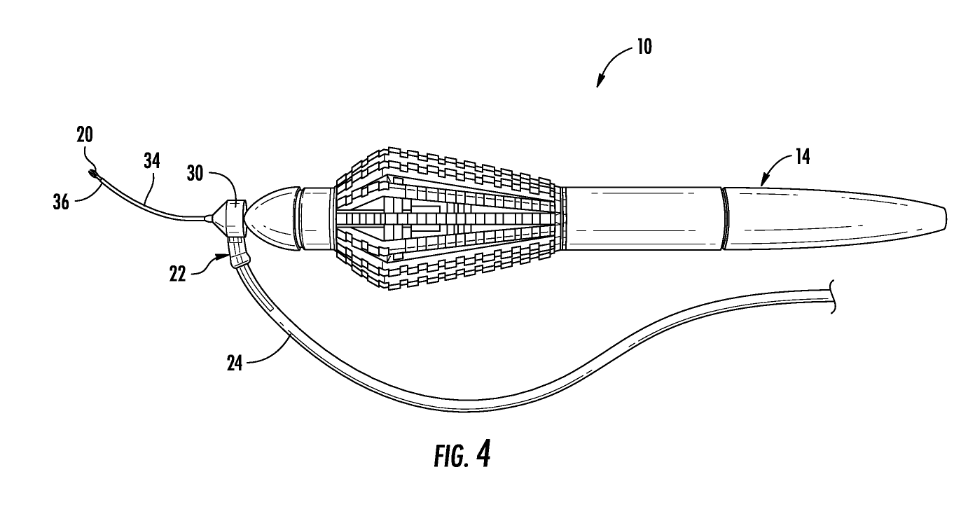

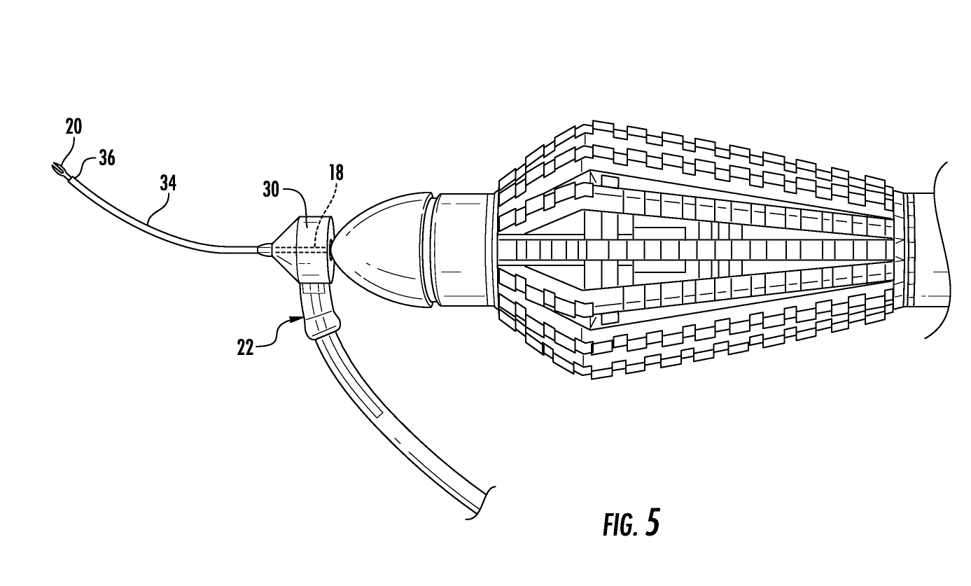

As shown in a variety of views in FIGS. 2A-5, a surgical tool assembly 10 is provided. The surgical tool assembly 10 is preferably used for Descemet membrane endothelial keratoplasty (DMEK) surgery. One of ordinary skill in the art would recognize from this disclosure that the surgical tool assembly 10 can used for other surgeries. The surgical tool assembly 10 includes a forceps instrument 12 with a proximal handle portion 14 including an actuator 16, and a distal forceps portion 18 including jaws 20. In one embodiment, the distal forceps portion 18 has a pre-formed curvature; however, one of ordinary skill in the art would recognize that the distal forceps portion 18 could have no curvature or be made of a bendable material that can curve, without departing from the spirit or subject matter of this application. The actuator 16 is configured to selectively move the jaws 20 between an open position and a closed or gripped position. In one embodiment, gripping the actuator 16 closes the jaws 20, and the jaws 20 have a default open position when the actuator 16 is not engaged or gripped. One of ordinary skill in the art would recognize from the present disclosure that other types of actuators 16 and jaws 20 can be used, without departing from the spirit or subject matter of this application.

An infusion assembly 22 includes a tube 24 configured to be connected to a fluid source 26 (shown schematically) at a primary tube end 28 and connected to a fitting 30 at a secondary or paracentesis tube end 32. In one embodiment, the fluid provided by the fluid source 26 is saline. One of ordinary skill in the art would recognize from the present disclosure that other fluids can be used for the surgical procedure and also based on the operator's preference. The fitting 30 defines an internal passage configured to receive the distal forceps portion 18 of the forceps instrument 12. In one embodiment, the passage of the fitting 30 is press-fit onto the distal forceps portion 18. One of ordinary skill in the art would recognize from the present disclosure that the fitting 30 can be attached to the distal forceps portion 18 in a variety of ways. The present embodiments disclosed herein provide a combined tool assembly 10 wherein the forceps instrument 12 and the infusion assembly 22 are connected to each other.

An infusion sleeve 34 extends away from the fitting 30, and defines an inner passage, such that the infusion sleeve 34 can be arranged concentric around an outer surface of the distal forceps portion 18 to define a fluid pathway. The jaws 20 extend beyond a terminal end 36 of the infusion sleeve 34 such that the jaws 20 are arranged outside of the infusion sleeve 34. In one embodiment, the inner diameter of the infusion sleeve 34 is 470-600 microns, and is more preferably 520-550 microns. In one embodiment, an outer diameter of the distal forceps portion 18 in a region away from the jaws 20 is 300-500 microns. The infusion assembly 22 provides fluid from the fluid source 26 to the terminal end 36 of the infusion sleeve 34 directly to a region of the jaws 20. Due to the infusion sleeve 34 concentrically surrounding the distal forceps portion 18, the infusion sleeve 34 provides pressurized fluid 360° around the jaws 20. In one embodiment, the jaws 20 having a gripping force capable of reliably holding a weight of 20 grams in suspension. In one embodiment, fluid provided to the terminal end 36 of the infusion sleeve 34 is at 1.0-30.0 psi. The gripping force of the jaws 20 is greater than a pressure level of fluid from the fluid source 26 to the infusion sleeve 34 to provide a reliable arrangement for securing a donor tissue 40 in position while injecting fluid around the jaws 20 to unscroll a donor tissue 40.

In one embodiment, the surgical tool assembly 10 includes a donor tissue preparation tool 38 configured to support a donor tissue 40. In one embodiment, the donor tissue preparation tool 38 is a spoon-like tool including a circular head portion 42 for supporting the donor tissue 40. As shown in FIGS. 2A and 2B, a groove 41 is provided on the donor tissue preparation tool 38 in one embodiment. The groove 41 is provided to reliably position the donor tissue 40 in a central portion of the donor tissue preparation tool 38. The groove 41 also functions to allow better ability to grasp an edge of the tissue with the forceps prior to pulling the tissue into the eye. In other embodiments, the donor tissue preparation tool 38 is a funnel guide tool, a spatula tool, forceps, or other known surgical tool capable of holding the donor tissue 40. For example, in one embodiment the donor tissue preparation tool 38 is a funnel guide, such as a Busing funnel guide (as disclosed in: https://www.surgicalinstruments.com/browse-by-type/product/16562-busin-glide-for-corneal-endo-implantation, [last visited Oct. 16, 2017] which is incorporated by reference as if fully set forth herein). One of ordinary skill in the art would recognize from the present disclosure that alternative types of donor tissue preparation tools can be used within the spirit of the subject matter of this application.

In one embodiment, the infusion assembly 22 is disposable, and the forceps instrument 12 and the donor tissue preparation tool 38 are reusable. As used herein, the term “disposable” with respect to the components of the assembly 10 indicates a component that is relatively inexpensive such that it is economically feasible to be discarded after a single use. In contrast, the term “reusable” indicates a component that is relatively expensive such that it is not economically feasible to be discarded after a single use. By providing an assembly 10 wherein a portion of the components are disposable, i.e. the infusion assembly 22, the overall costs of the assembly 10 is reduced. In another embodiment, the infusion assembly 22 and the donor tissue preparation tool 38 are disposable. In one embodiment, the infusion assembly 22 is formed from plastic. In one embodiment, the donor tissue preparation tool 38 is formed from metal, and is preferably formed from steel or titanium.

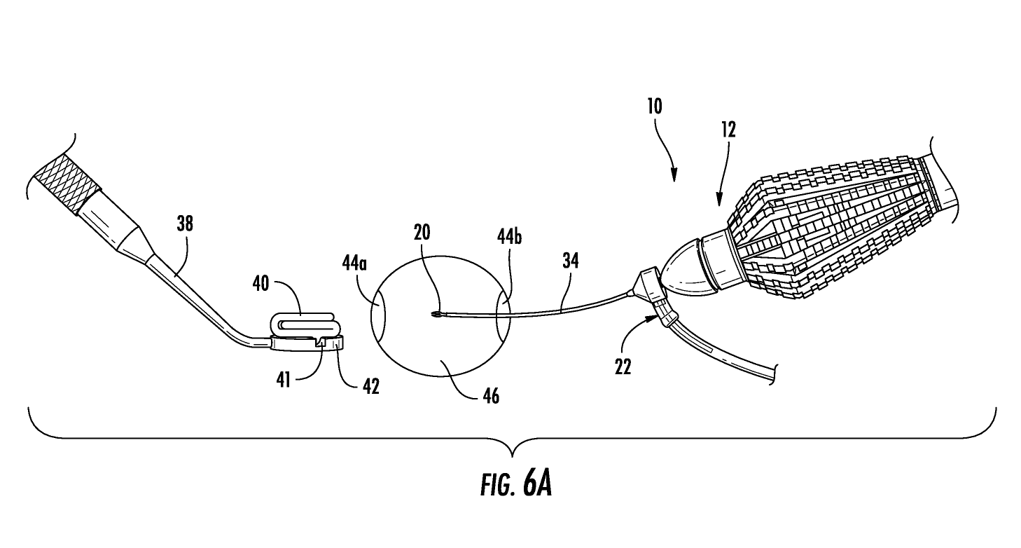

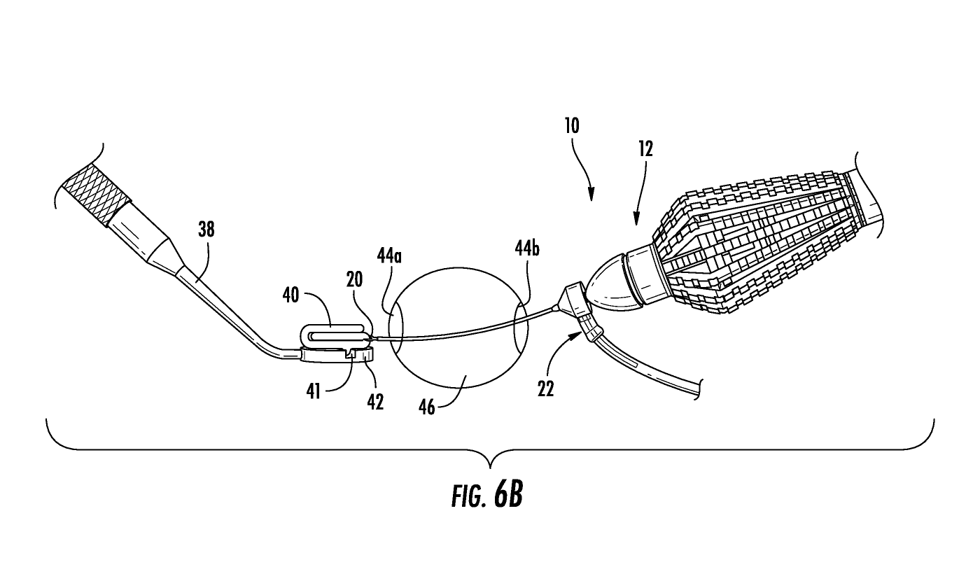

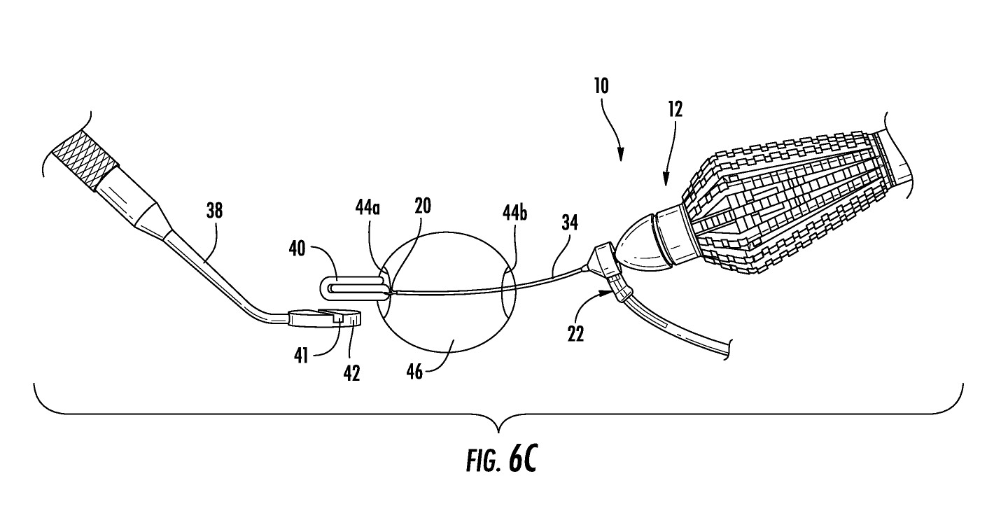

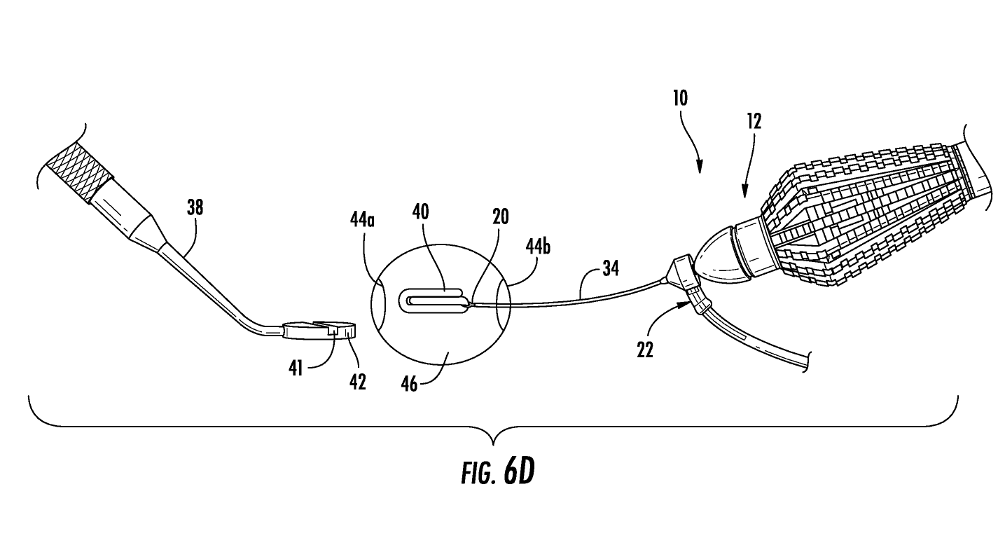

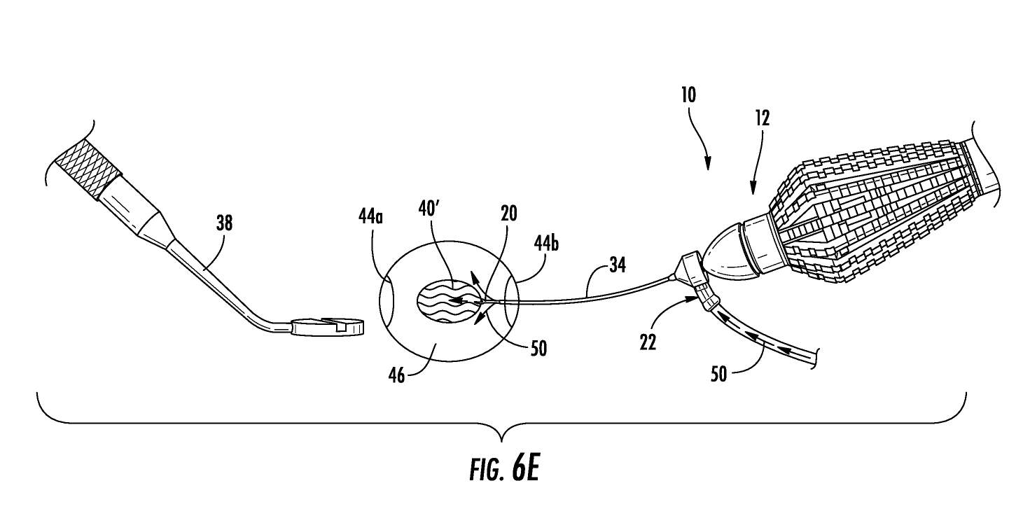

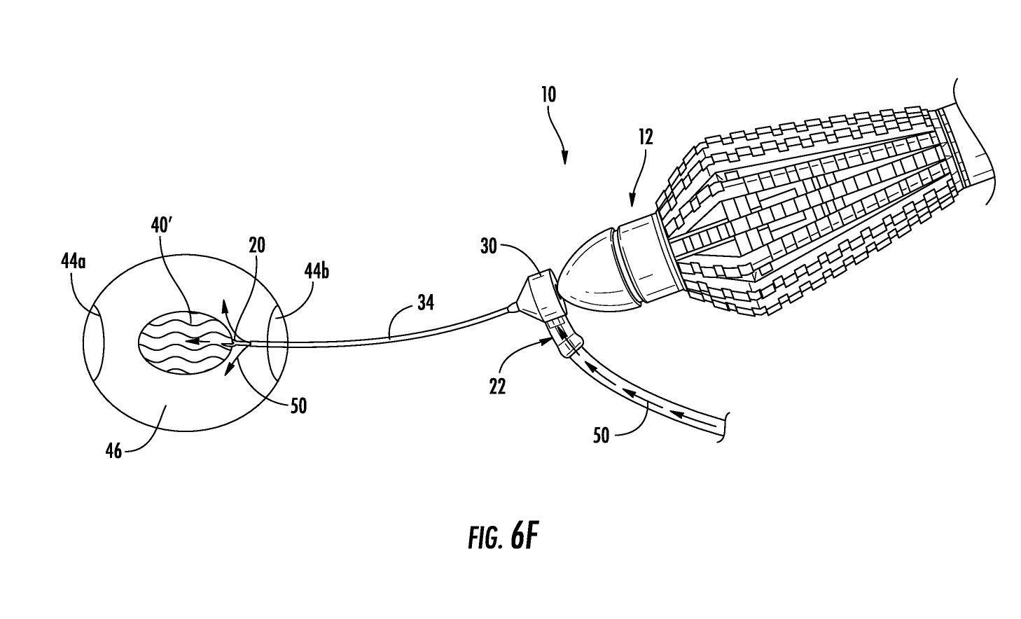

In one embodiment, a method of performing corneal surgery is provided. In an embodiment the corneal surgery is DMEK surgery. FIGS. 6A-6F illustrate the steps of performing corneal surgery. The method includes providing the surgical tool assembly 10 described above including the forceps instrument 12, the infusion assembly 22, and the donor tissue preparation tool 38. The method includes forming two incisions 44a, 44b (i.e. a primary incision 44a of 2.0-2.5 mm and a paracentesis incision 44b of 1.0-1.5 mm) into the anterior chamber of the patient's eye 46. The incisions are made in the cornea, and allow access to the anterior chamber. These incisions can be made using any known cutting method. As shown in the drawings, the two incisions 44a, 44b are on diametrically opposed hemispheres of the anterior chamber of the patient's eye 46. Each of the incisions 44a, 44b are less than 2.5 mm. In one embodiment, incision 44a has a length of 2.0-2.5 mm. In one embodiment, incision 44b has a length of 1.0-1.5 mm. In one embodiment, the primary incision 44a is larger than the paracentesis incision 44b. Neither of the incisions 44a, 44b requires a suture to be closed due to the relatively small dimensions of the incisions 44a, 44b. One of ordinary skill in the art recognizes that an original cornea tissue can be removed from the anterior chamber of the patient's eye 46 during any step prior to implanting the donor tissue 40 in the anterior chamber of the patient's eye 46.

The method includes placing the donor tissue 40 on the donor tissue preparation tool 38 in a trifold orientation with endothelium inward and positioning the donor tissue preparation tool 38 adjacent to the primary incision 44a. As shown in FIG. 6A, the circular head portion 42 of the donor tissue preparation tool is directly adjacent to the primary incision 44a of the anterior chamber of the patient's eye 46. As shown in FIG. 6B, the method includes inserting the distal forceps portion 18 of the forceps instrument 12 through the paracentesis incision 44b, through the anterior chamber of the patient's eye 46, and out of the primary incision 44a. As shown in FIG. 6C, the method includes securing the donor tissue 40 from the donor tissue preparation tool 38 via the jaws 20. As shown in FIG. 6D, the method includes withdrawing the distal forceps portion 18 back through the primary incision 44a and into the anterior chamber of the patient's eye 46 at an installation site. In one embodiment, this step includes gripping a circumferential edge of the donor tissue 40 with the jaws 20.

Although the orientation of the donor tissue preparation tool 38 and the donor tissue 40 are shown in one orientation in FIGS. 6A-6E, one of ordinary skill in the art would recognize from the present disclosure that the orientation of these components can change relative to the patient's eye. For example, the orientation of the donor tissue preparation tool 38 and the donor tissue 40 can be rotated 90 degrees relative to the patient's eye. In one embodiment, a longitudinal axis of the folded tissue 40 can be aligned with the groove 41 of the donor tissue preparation tool 38.

In one embodiment, the donor tissue 40 has an outer diameter that is greater than a length of the incisions 44a such that the donor tissue 40 folds (i.e. scrolls) onto itself when being pulled back through the primary incision 44a. This ensures that the incisions 44a and 44b have minimal dimensions, and therefore minimizes trauma to the anterior chamber of the patient's eye 46. A surgeon or medical professional can manipulate the surgical tool assembly 10 by selectively gripping the actuator 16 to grab the donor tissue 40 with the jaws 20. By inserting the distal forceps portion 18 in one incision 44b and out of the other incision 44a, and then pulling the gripped donor tissue 40 backwards into the anterior chamber of the patient's eye 46 through the primary incision 44a, this method avoids “injecting” the donor tissue 40 into the anterior chamber of the patient's eye 46, which typically requires a larger incision.

As shown in FIGS. 6E and 6F, the method includes providing pressurized fluid 50 to the fluid pathway of the infusion sleeve 34 while gripping the donor tissue 40 to orient and unscroll the donor tissue 40 (shown in the unscrolled state as donor tissue 40′) at the installation site inside the anterior chamber of the patient's eye 46. This method provides a much faster and more reliable configuration to unscroll the donor tissue 40′ within the anterior chamber of the patient's eye 46. According to one embodiment, the method can unscroll the donor tissue 40′ in less than sixty seconds. While holding the donor tissue in position, a small amount of fluid is aspirated to shallow, i.e. to decrease the volume of, the anterior chamber of the patient's eye to prevent the donor tissue from scrolling. In one embodiment, a plunger of a syringe is driven in a first direction to infuse the pressurized fluid into the anterior chamber of the patient's eye. After the pressurized fluid is provided, the aspiration step is performed by a user withdrawing the plunger of the syringe, which effectively decreases the volume of the anterior chamber of the patient's eye and helps set the donor tissue in position. One of ordinary skill in the art would understand that the aspiration step can be performed by a variety of configurations.

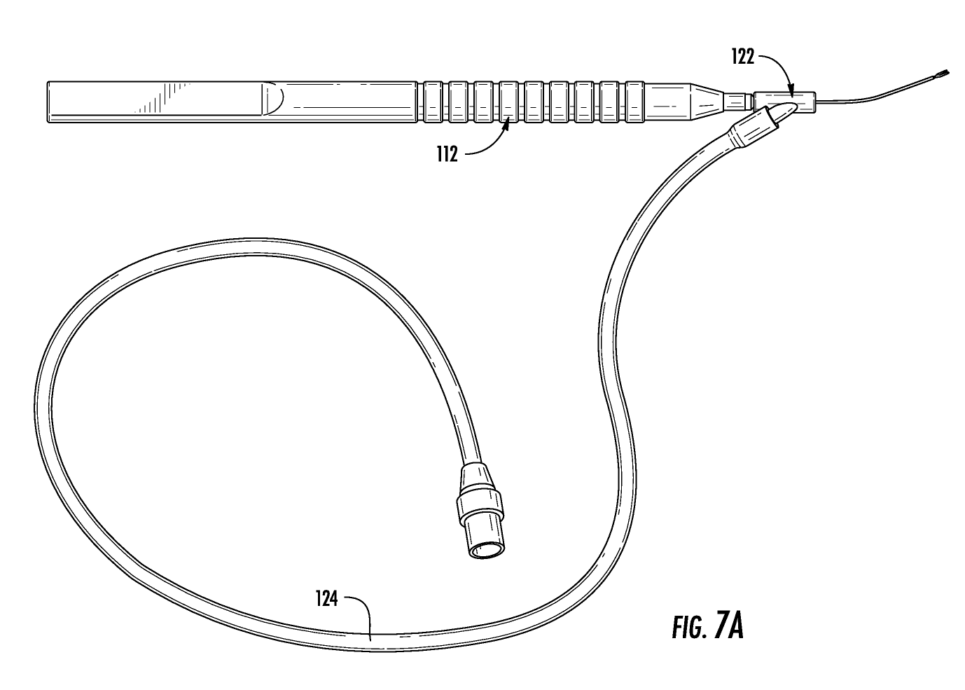

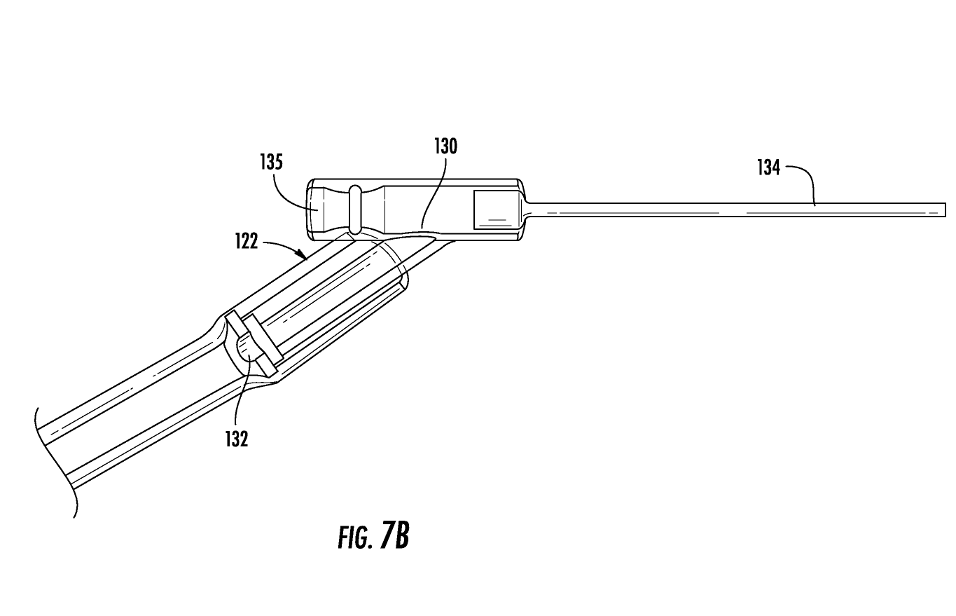

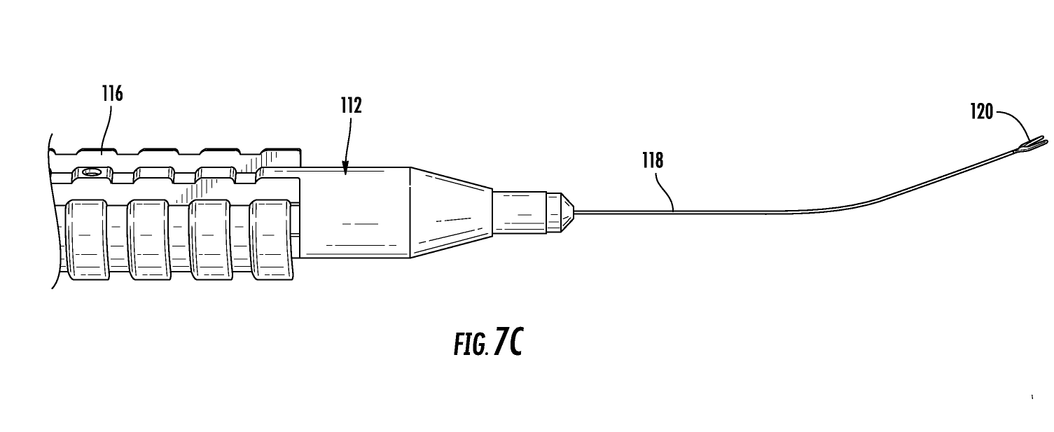

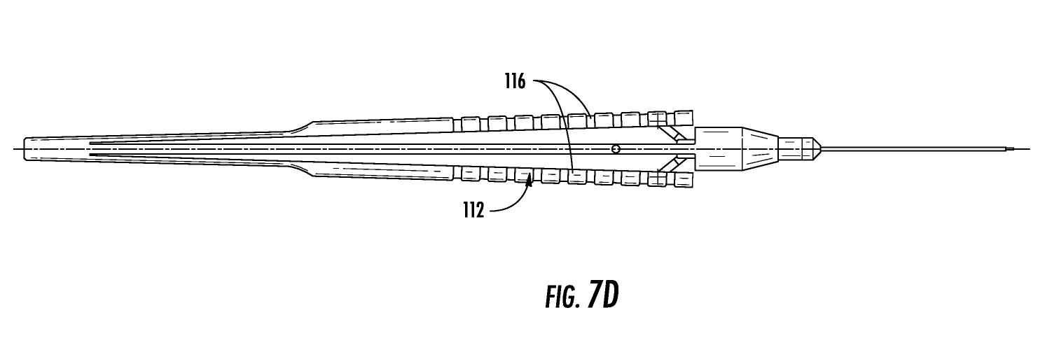

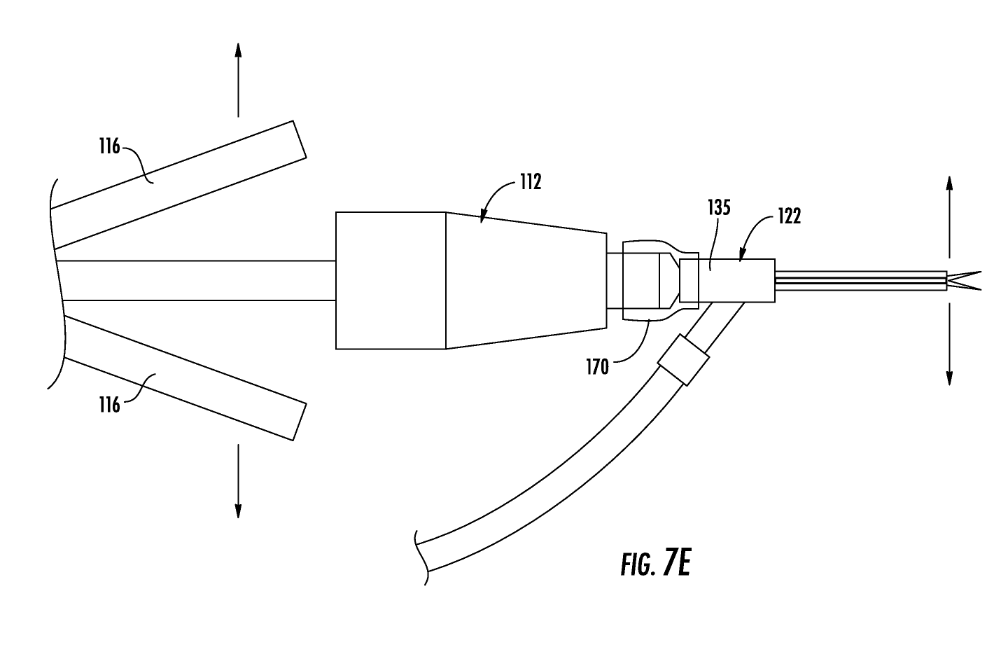

FIG. 7A-7E illustrate an alternative embodiment of the surgical tool assembly. The embodiment shown in FIGS. 7A-7E is functionally identical to the features described herein with respect to the other embodiments. As shown in FIG. 7A, the handle of the forceps instrument 112 is different than the forceps instrument 12 illustrated in the other drawings. The handle includes two wings or prongs for the actuator 116, best shown in FIGS. 7D and 7E. The infusion assembly 122 includes a fitting 130 with a tubular engagement portion 135 that is adapted to engage around an outer surface of the forceps instrument 112. The secondary or paracentesis tube end 132 and the infusion sleeve 134 perform the same function as the corresponding parts described herein. The forceps instrument 112 includes a distal forceps portion 118 with jaws 120.

A secondary sealing component 170 between the forceps instrument 112 and the infusion assembly 122 is illustrated in FIG. 7E. A sleeve 170 is provided between the forceps instrument 112 and the infusion assembly 122, and wraps around an outer surface of each of these components. The sleeve 170 provides a reliable seal between the forceps instrument 112 and the infusion assembly 122. Other features of the forceps instrument 112 and the infusion assembly 122 are functionally the same as the features described above with respect to the forceps instrument 12 and the infusion assembly 22.

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

What is claimed is:

1. A surgical tool assembly comprising:
a forceps instrument including a proximal handle portion with an actuator, and a distal forceps portion including jaws, where the actuator selectively moves the jaws; and
an infusion assembly including a tube configured to be connected to a fluid source at a primary tube end and connected to a fitting at a paracentesis tube end, the fitting defines a passage configured to receive the distal forceps portion of the forceps instrument, an infusion sleeve extending away from the fitting, the infusion sleeve being arranged concentric around an outer surface of the distal forceps portion to define a fluid pathway, and the jaws extend beyond a terminal end of the infusion sleeve such that the jaws are arranged outside of the infusion sleeve.

2. The surgical tool assembly of claim 1, further comprising a donor tissue preparation tool configured to support a donor tissue.

3. The surgical tool assembly of claim 2 wherein the donor tissue preparation tool has a circular spoon to hold the prepared donor tissue.

4. The surgical tool assembly of claim 1, wherein the surgical tool assembly is used for Descemet membrane endothelial keratoplasty (DMEK) surgery.

5. The surgical tool assembly of claim 1, wherein the distal forceps portion has a pre-formed curvature.

6. The surgical tool assembly of claim 1, wherein the actuator is configured to grip the jaws when the actuator is squeezed.

7. The surgical tool assembly of claim 1, wherein fluid is provided via the tube to a terminal end of the infusion sleeve at 1.0 psi-30.0 psi.

8. The surgical tool assembly of claim 1, wherein the passage of the fitting is press-fit onto the distal forceps portion.

9. The surgical tool assembly of claim 1, further comprising a sleeve adapted to engage around an outer surface of both the infusion assembly and the forceps instrument to provide a sealed connection.

10. A method of performing corneal surgery, the method comprising:
(a) providing a surgical tool assembly including:
(i) a forceps instrument including a proximal handle portion with an actuator, and a distal forceps portion including jaws, where the actuator selectively moves the jaws;
(ii) an infusion assembly including a tube configured to be connected to a fluid source at a first tube end and connected to a fitting at a second tube end, an infusion sleeve extending away from the fitting, the fitting defines a passage configured to receive the distal forceps portion of the forceps instrument, the infusion sleeve being arranged concentric around an outer surface of the distal forceps portion to define a fluid pathway, and the jaws extend beyond a terminal end of the infusion sleeve such that the jaws are arranged outside of the infusion sleeve; and
(b) cutting two incisions on the anterior chamber of the patient's eye;
(c) placing the donor tissue adjacent to a primary incision;
(d) inserting the distal forceps portion of the forceps instrument through a paracentesis incision through the anterior chamber of the patient's eye and out of the primary incision;
(e) securing the donor tissue via the jaws and withdrawing the distal forceps portion back through the primary incision and into the anterior chamber of the patient's eye at an installation site;
(f) providing pressurized fluid to the fluid pathway of the infusion sleeve while gripping the donor issue to orient and unscroll the donor tissue at the implantation site; and
(g) while holding the donor tissue in position, an amount of fluid is aspirated to shallow the anterior chamber of the patient's eye to prevent the donor tissue from scrolling.

11. The method of claim 10, further comprising:
providing a donor tissue preparation tool configured to support the donor tissue; and
placing the donor tissue in a scrolled state on the donor tissue preparation tool during step (c).

12. The method of claim 10, wherein the two incisions of step (b) are on diametrically opposed halves of the anterior chamber of the patient's eye.

13. The method of claim 10, wherein the amount of fluid in step (g) is 0.010 ml to 1.0 ml.

14. The method of claim 10, wherein step (e) includes gripping a circumferential edge of the donor tissue.

15. The method of claim 10, wherein the corneal surgery is keratoplasty surgery.

16. The method of claim 15, wherein the keratoplasty surgery is Descemet membrane endothelial keratoplasty (DMEK).

17. The method of claim 16, wherein a patient's existing Descemet membrane is removed prior to step (d).

18. The method of claim 10, wherein the donor tissue is Descemet membrane and endothelial tissue.

19. The method of claim 10, wherein the primary incision and the paracentesis incision each have a length less than 2.5 mm.

20. The method of claim 10, wherein the primary incision has a length of 2.0 mm to 2.5 mm and the paracentesis incision has a length of 1.0 mm to 1.5 mm.

21. A method of positioning and placing corneal donor tissue in the anterior chamber of a patient's eye, the method comprising:
(a) providing a surgical tool assembly including:
(i) a forceps instrument including a proximal handle portion with an actuator, and a distal forceps portion including jaws, where the actuator selectively moves the jaws;
(ii) an infusion assembly including a tube configured to be connected to a fluid source at a primary tube end and connected to a fitting at a paracentesis tube end, an infusion sleeve extending away from the fitting, the fitting defines a passage configured to receive the distal forceps portion of the forceps instrument, the infusion sleeve being arranged concentric around an outer surface of the distal forceps portion to define a fluid pathway, and the jaws extend beyond a terminal end of the infusion sleeve such that the jaws are arranged outside of the infusion sleeve; and
(iii) a donor tissue preparation tool configured to support a donor tissue;
(b) placing donor tissue on the donor tissue preparation tool;
(c) securing the donor tissue from the donor tissue preparation tool via the jaws and positioning the donor tissue into the anterior chamber of the patient's eye at an installation site;
(d) providing pressurized fluid to the fluid pathway of the infusion sleeve while gripping the donor issue to orient and unscroll the donor tissue at the installation site; and
(e) while holding the donor tissue in position, a small amount of fluid is aspirated to shallow the anterior chamber of the patient's eye to prevent the donor tissue from scrolling.

22. A configuration of a surgical tool securing a donor tissue, the configuration comprising
(a) an assembly comprising:
a forceps instrument including a proximal handle portion with an actuator, and a distal forceps portion including jaws, where the actuator selectively moves the jaws; and
an infusion assembly including a tube configured to be connected to a fluid source at a primary tube end and connected to a fitting at a paracentesis tube end, the fitting defines a passage configured to receive the distal forceps portion of the forceps instrument, an infusion sleeve extending away from the fitting, the infusion sleeve being arranged concentric around an outer surface of the distal forceps portion to define a fluid pathway, and the jaws extend beyond a terminal end of the infusion sleeve such that the jaws are arranged outside of the infusion sleeve; and
(b) a donor tissue.
